(19) United States
(12) Patent Application Publication
(10) Pub. No.: US 2006/0104967 A1
(43) Pub. Date: May 18, 2006

(54) Method of converting the state of proteins

(76) Inventors: Akira Yamada, Koganei (JP); Kazuhiro Oiwa, Koganei (JP)

Correspondence Address: SNELL & WILMER LLP, 600 ANTON BOULEVARD, SUITE 1400, COSTA MESA, CA, 92626, US

(21) Appl. No.: 11/186,023

(22) Filed: Jul. 21, 2005

(30) Foreign Application Priority Data
Nov. 15, 2004 (JP) P2004-331159

(51) Int. Cl. A61K 38/46

(52) U.S. Cl. 424/94.6

(57) ABSTRACT

Since the mechanism of conversion of the state of actin and myosin or the filaments containing myosin from the relaxed to the catch had not been totally clear and its reconstitution was not easily carried out, a method of converting the state of the muscle proteins from the relaxed state, where said actin does not bind to myosin or filaments containing myosin, to the catch state, where actin binds to myosin or the filaments containing myosin and the binding can sustain a certain tension with low energy expenditure, is accomplished by using a phosphatase called serine/threonine protein phosphatase 2B dephosphorylating twitchin. The phosphatase forms thick filaments along with myosin, and at the same time, the state is converted from the catch state to the relaxed state by using a protein kinase A phosphorylating twitchin, and thus the state can be reversibly converted between the catch and the relaxed states.

### BACKGROUND OF THE INVENTION

1. Field of the Invention

The present invention relates to a method of converting the state of muscle proteins, containing actin, myosin, and twitchin between a catch and a release state.

2. Description of Related Art

Conventionally, it has been known that there is a state called “catch” where muscles maintain high tension with low energy expenditure by the binding of actin filaments to myosin filaments in addition to an active state where muscles contract by the sliding movement of myosin filaments against actin filaments and the relaxed state where muscles do not produce significant tension due to detachment of actin filaments from myosin filaments. The present inventors successfully reconstituted the above catch and relaxed states in vitro by using native filaments or purified proteins obtained from so called “catch muscles”, see Proceedings of National Academy of Sciences Jun. 5, 2001 vol. 98; pp 6635-6640 and Genetics September 2001.

In contrast, it has already been revealed that the catch state and the relaxed state can reversibly be converted to each other, in that the phosphorylation of a protein called twitchin, which forms the thick filaments together with myosin, is involved in the relaxation of the catch, and that the dephosphorylation of this protein is involved in the initiation of the catch state, based on studies using skinned muscle cells.

### SUMMARY OF THE INVENTION

As for the reconstitution of the relaxed state, an enzyme responsible for the phosphorylation of twitchin has already been identified and its regulatory mechanism been revealed, whereas the regulatory mechanism of conversion from the relaxed state to the catch state has not yet been well characterized and understood.

Therefore, the present inventors, as a result of effort and study, have successfully identified a phosphatase responsible for the initiation of the catch state of the muscles and have developed a simple method to artificially create the catch state.

The present invention is a state conversion method of muscle proteins containing actin, myosin and twitchin, which form thick filaments together with myosin, as well as a protein state conversion method to convert from the relaxed state, where said actin does not bind to myosin or the filaments containing myosin, to the catch state, where actin binds to myosin or the filaments containing myosin, maintaining a certain tension in muscles, using a phosphatase called serine/threonine protein phosphatase 2B to dephosphorylate twitchin. Here, “myosin or the filaments containing myosin” indicate the state of myosin forming filaments, myosin not forming filaments, and myosin forming filaments along with twitchin or other associated proteins.

In addition, as another preferred embodiment of the present invention, there are state conversion methods of the muscle proteins containing actin, myosin and twitchin to convert from the relaxed state, where actin does not bind to myosin or the filaments containing myosin, to the catch state, where actin binds to myosin or the filaments containing myosin, which can maintain high tension with low energy expenditure, by using serine/threonine protein phosphatase 2B to dephosphorylate twitchin, and to convert from the catch state to the relaxed state by using protein kinase A to phosphorylate twitchin, and thus to reversibly convert the states between catch and relaxed.

It is desirable to use approximately 10⁻⁵M free Ca²⁺ when converting the state to catch.

Calcineurin can be used as a serine/threonine protein phosphatase 2B. For instance, bovine calcineurin, as it is easy to obtain, can be used. In this case, it is recommended to add a certain amount of calmodulin (for instance, adding 2 mM calmodulin when 0.2 mM calcineurin is used) to fully activate calcineurin.

According to the present invention, the catch state can artificially and easily be created by using actin, myosin and twitchin, and the resulting catch binding can sustain high tension with low energy expenditure.

As a result, a simple technique of conversion between the relaxed and the catch states has been achieved by combining a conversion procedure from the catch to the relaxed state which has already been established and another conversion procedure from the relaxed to the catch state which has been enabled in the present invention.

Furthermore, it will be possible to develop new devices which can be used in nano machines by taking advantage of this regulatory mechanism of the relaxed and the catch states created with actin, myosin and twitchin.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 shows an outline explanatory diagram of one embodiment of the present invention,

FIG. 2 shows pictures representing the state of the protein in the embodiment,

FIG. 3 shows pictures representing the state of the protein in the embodiment,

FIG. 4 shows pictures representing the state of the protein in the embodiment,

FIG. 5 shows pictures representing the state of the protein in the embodiment,

FIG. 6 shows pictures representing the state of the protein in the embodiment,

FIG. 7 shows pictures representing the state of the protein in the embodiment,

FIG. 8 shows views representing results of an SDS-PAGE and autoradiography of myosin and twitchin in the embodiment,

FIG. 9 shows experimental images supporting the present invention,

FIG. 10 shows experimental images supporting the present invention, and

FIG. 11 shows experimental images supporting the present invention.

### DETAILED DESCRIPTION OF THE PREFERRED EMBODIMENTS

Reference will now be made in detail to the preferred embodiments of the invention which set forth the best modes contemplated to carry out the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and circuits have not been described in detail as not to unnecessarily obscure aspects of the present invention.

A preferred embodiment of the present invention is described hereafter, with reference to the figures.

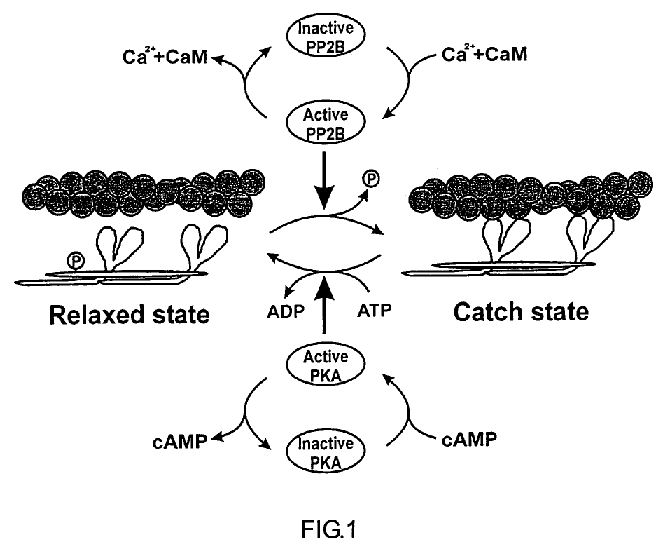

The present state conversion method is a method to convert the state of muscle proteins containing actin, myosin and twitchin from a relaxed state, where actin does not bind to myosin or the filaments containing myosin, to a catch state, where actin binds to myosin or the filaments containing myosin, where the binding can sustain high tension, by using serine/threonine protein phosphatase 2B (described as PP2B hereafter) to dephosphorylate twitchin, as well as to reversibly convert the state from catch to relaxed by phosphorylating twitchin by using protein kinase A (described as PKA hereafter). The model of these reversible changes is shown in FIG. 1.

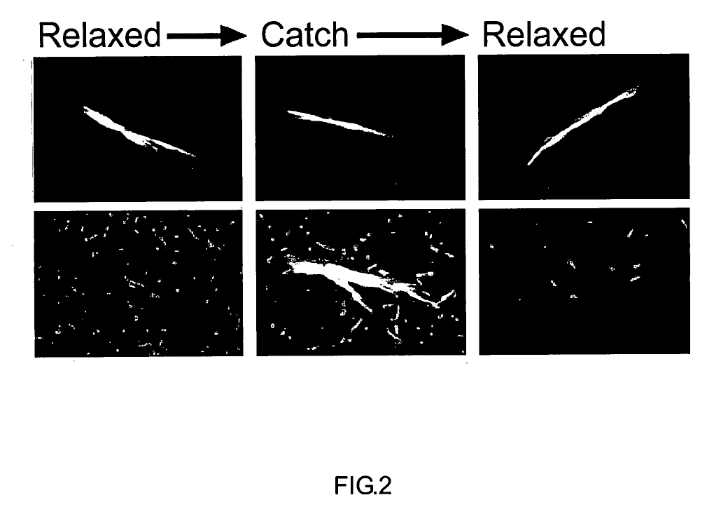

These reversible changes of the states are shown in pictures represented in FIG. 2. The upper and lower pictures in FIG. 2 respectively show dark-field and fluorescence images. Synthetic thick filaments containing myosin and twitchin were observed in the dark-field images and the fluorescent actin filaments, which will be described later, were observed in the fluorescence images.

Actin used in the present study was purified from rabbit skeletal muscles and labeled with fluorescence. More specifically, actin filaments were labeled with tetramethylrhodamine phalloidin (Sigma, P-1951) and the concentration was 2 μg/ml. In addition, native thin filaments obtained from the homogenate of the byssus retractor and posterior adductor muscles (smooth muscles), namely catch muscles of M. galloprovincialis, can be used, but it has already been proven by the present inventors that they can be substituted by purified actin filaments prepared from rabbit skeletal muscles.

As described above, the states when the synthetic thick filaments were used are shown in FIG. 2. However we used myosin purified from the above catch muscles of M. galloprovincialis in a prescribed manner in the present embodiment. The purification method includes, for instance, performing gel filtration chromatography of 40-60% ammonium sulfate fraction of high salt extract of the muscle homogenate. In the present embodiment, a Superose 6 HR 10/30 column (Amersham Pharmacia) was used for the gel filtration chromatography.

Twitchin used in the present study was purified in a prescribed manner from the same muscles. The purification method includes, for instance, performing anion exchange chromatography of 30-40% ammonium sulfate fraction of high salt extract of the muscle homogenate after dissolving a buffer solution containing 0.15M potassium phosphate, 1 mM EGTA, 1 mM MgCl₂ and 2 mM DTT (pH7.5). In the present embodiment, a Mono Q HR 5/5 column (Amersham Pharmacia) was used for the anion exchange chromatography and twitchin was eluted with 0.15M KCl in the same solution.

For the dephosphorylation of twitchin, either the soluble protein fraction of catch muscles containing PP2B or bovine calcineurin classified as a PP2B was used in the present embodiment.

For the phosphorylation of twitchin, either the soluble protein fraction of catch muscles containing PKA or bovine PKA was used in the present embodiment.

The soluble protein fraction used in the present study was prepared as follows. First, the suspension was obtained by homogenizing catch muscles of M. galloprovincialis in a certain buffer solution, containing 80 mM NaCl, 2 mM MgCl₂, 0.5 mM EGTA, 2 mM DTT, and 20 mM Pipes-NaOH, adjusted to pH 7.0 in the present embodiment. Then, the supernatant was obtained by centrifuging (for instance, at 300,000×g for 30 min.) and used for the study.

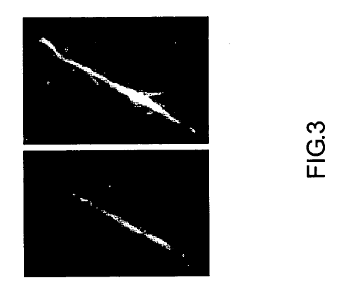

The steps to initiate the catch state of muscles using the actin, myosin, and twitchin described above and the steps to reversibly reconstitute the catch and relaxed states using PP2B and PKA will be described hereafter, with reference to FIGS. 3 through 7. Furthermore, in FIGS. 3 through 7, left and right pictures respectively show dark-field and fluorescence images in the same field of view.

First, the reconstitution of the catch and the relaxed states using the soluble protein fraction will be described. The protein concentration of the soluble protein fraction used in the present embodiment was 0.2 mg/ml.

Twitchin was mixed with the soluble protein fraction in the presence of 10⁻⁵M free Ca²⁺ (This treatment is described as “catch treatment” hereafter). After incubation at room temperature (24° C.) for an hour, an excess amount of EGTA was added to lower the free Ca²⁺ to less than 10⁻⁷M.

Myosin filaments were obtained by dialyzing purified myosin, and were mixed with twitchin after the above catch treatment. When the resulting myosin filaments were mixed with fluorescence-labeled actin filaments in the presence of Mg-ATP at a low free Ca²⁺ concentration, they bound to actin filaments (See FIG. 3).

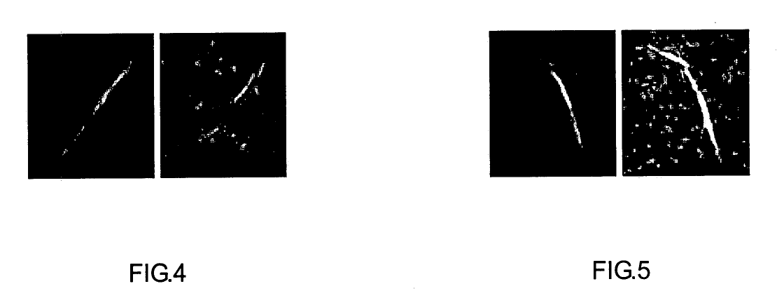

In contrast, when twitchin was mixed with the soluble protein fraction in the presence of 10⁻⁵M cAMP and 1⁻⁴ mM ATP (This treatment is described as “relaxation treatment” hereafter), actin filaments did not bind to the thick filaments containing twitchin and myosin (See FIG. 4).

Further catch treatment with the soluble protein fraction resulted in binding of actin filaments to the thick filaments (See FIG. 5).

In addition, the following results were obtained when bovine PKA and bovine calcineurin, instead of said soluble protein fraction, were used for phosphorylation and dephosphorylation of twitchin, respectively.

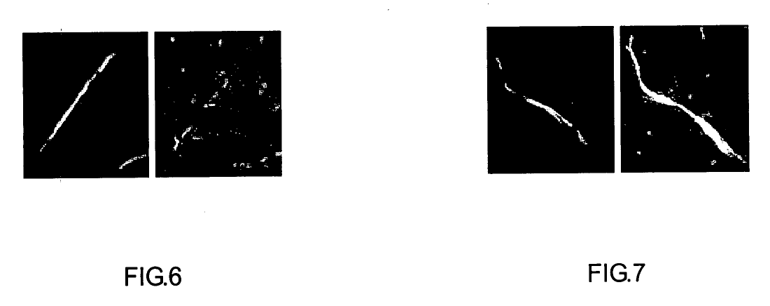

When 2 mg/ml bovine PKA (Sigma P-5511) was added in the presence of 10⁻⁵M cAMP and 1⁻⁴ mM ATP to the thick filaments in the catch state such as shown in FIG. 3, the relaxed state was obtained as shown in FIG. 6.

Then, the thick filaments in the relaxed state were further mixed with 0.2 mM bovine calcineurin (Sigma C-1907) and 2 mM calmodulin (Sigma P-2277) in the presence of 10⁻⁵M free Ca²⁺. After the incubation for 10 minutes at room temperature (24° C.), the excess amount of EGTA was added to lower the free Ca²⁺ concentration to less than 10⁻⁷M. When actin filaments were added, they bound to the thick filaments as shown in FIG. 7.

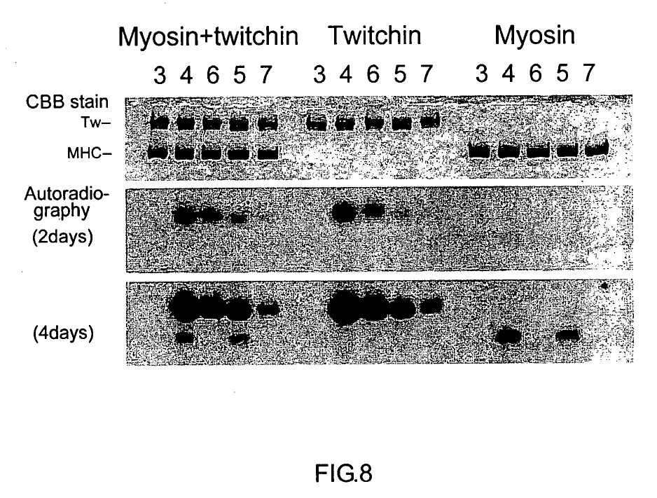

When the phosphorylation levels of twitchin (Tw) and myosin heavy chain (MHC) after the relaxation treatment and further catch treatment as described above were individually analyzed by an SDS-PAGE and autoradiography by using [γ⁻³²P]ATP, the results were obtained as shown in FIG. 8. Numerical symbols, such as 3, 4, 6, 5 and 7 in FIG. 8 respectively indicate the states of myosin and twitchin after the catch treatment by the soluble protein fraction (FIG. 3), after further relaxation treatment by the soluble protein fraction in the presence of [γ⁻³²P]ATP (FIG. 4), the relaxation treatment by bovine PKA in the presence of [γ⁻³²P]ATP (FIG. 6), after the catch treatment of the proteins in state 4 by the soluble protein fraction (FIG. 5) and after the catch treatment of the proteins in state 6 by bovine calcineurin (FIG. 7).

As shown in lanes 4 and 6 in FIG. 8, it is clear that twitchin was phosphorylated by the relaxation treatment with either the soluble protein fraction or bovine PKA. Myosin was slightly phosphorylated by the relaxation treatment with the soluble protein fraction, but not phosphorylated at all by the relaxation treatment with bovine PKA, indicating that this slight phosphorylation of myosin was due to other enzymes than PKA.

In addition, as shown in lanes 5 and 7 in FIG. 8, twitchin was dephosphorylated by the catch treatment with either the soluble protein fraction or bovine calcineurin.

The study performed by the present inventors revealing that the enzyme responsible for dephosphorylation of twitchin was PP2B will be explained below.

First, the inhibitory effects of phosphatase inhibitors, okadaic acid and microcystin LR, on the initiation of the catch state were investigated by changing their concentrations.

The concentrations used in this study were 3 mM, 10 mM, 50 mM and 100 mM for okadaic acid and 0.2 mM, 0.5 mM, 1 mM and 2 mM for microcystin LR.

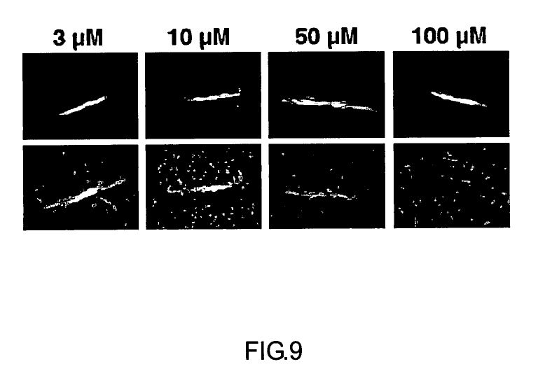

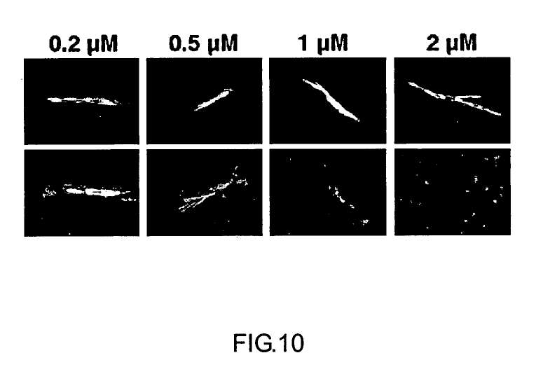

The inhibitory effects at various concentrations were observed with a dark-field microscope and a fluorescence microscope. The results are shown in FIGS. 9 and 10. In these figures, the upper and lower pictures respectively show dark-field and fluorescence images. Synthetic thick filaments containing myosin and twitchin were observed in the dark-field images, and the fluorescent actin filaments were observed in the fluorescence images.

Based on the observed inhibitory effects of various concentrations of inhibitors and the facts that Ki values for PP2B, concentrations of inhibitors where the enzyme activity becomes to half, are 5 mM for okadaic acid and 0.2 mM for microcystin LR, it was suggested that the phosphatase involved in the initiation of the catch was PP2B.

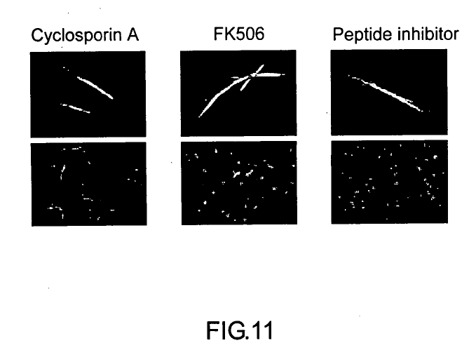

Then, the effects of inhibitors specific for PP2B, i.e., cyclosporin A, FK 506, and a calcineurin peptide inhibitor, were investigated. As shown in FIG. 11, actin filaments did not bind to the synthetic thick filaments at all when the catch treatment was performed in the presence of these inhibitors. These results confirmed that the phosphatase involved in the initiation of the catch state was PP2B. In FIG. 11, the upper and lower pictures respectively show dark-field and fluorescence images.

Furthermore, the observation that Ca²⁺ was required for the initiation of the catch state supports the idea that PP2B was involved since PP2B is known as a calcium-dependent phosphatase.

In addition, the result that bovine calcineurin and calmodulin, instead of the soluble protein fraction, can be used for the initiation of the catch state indicates that the factors responsible for the initiation of the catch state in the soluble protein fraction were just PP2B and its activator, calmodulin.

As described above, according to the protein state conversion method described in the present embodiment, the catch and the relaxed states can be reconstituted with purified actin, myosin, and twitchin by using either the soluble protein fraction containing essential enzymes obtained through a certain fraction method from catch muscles, or bovine calcineurin and bovine PKA.

These catch and relaxed states are respectively analogous to “a state in a brake position” and “a state in a neutral position” in automobiles, and therefore, the applications taking advantage of this regulatory mechanism to the development of new devices such as nano machines are highly expected.

In addition, this could serve as a model to study the “latch” mechanism of vertebrate smooth muscles such as vascular and intestinal muscles, which is apparently similar to the catch mechanism of molluscan muscles.

Furthermore, the conversion of the states between the catch and relaxed states can be accomplished by using commercially available enzymes, such as bovine calcineurin and bovine PKA.

Furthermore, the present invention is not limited to the method described in the present embodiment.

For instance, although the purified actin, myosin, and twitchin were used in the present embodiment, it is not always necessary to use purified proteins to achieve the catch and relaxed states in the present invention. For instance, such synthetic thick filaments containing myosin and twitchin as used in the above inhibitory study can be used. In addition, the purification method is not limited to the method described above. Furthermore, genetically engineered and/or modified proteins can be used.

The experimental conditions such as concentrations of various reagents and temperature in converting protein states are not limited to the conditions described in the above embodiment.

In addition, for PP2A and PKA, it is not necessary to use bovine PP2B and bovine PKA.

In addition, various detailed specifications in each part are not limited to the specifications described in the above embodiment and various modifications can be made without deviating from the spirit and scope of the present invention.

Those skilled in the art will appreciate that various adaptations and modifications of the just-described preferred embodiment can be configured without departing from the scope and spirit of the invention. Therefore, it is to be understood that, within the scope of the amended claims, the invention may be practiced other than as specifically described herein.

What is claimed is:

1. A method to convert the state of muscle proteins containing actin, myosin, and twitchin, which forms thick filaments along with the myosin, in which the state is converted from a relaxed state, where said actin does not bind to said myosin or the filaments containing myosin, to a catch state, where said actin binds to said myosin or the filaments containing myosin and the binding can sustain a certain tension comprising; applying a serine/threonine protein phosphatase 2B dephosphorylating twitchin.

2. The protein state conversion method of claim 1, wherein the conversion to said catch state is carried out at approximately 10⁻⁵M free Ca²⁺.

3. The protein state conversion method of claim 2, wherein calcineurin is used as said serine/threonine protein phosphatase 2B.

4. The protein state conversion method of claim 3, wherein a predetermined amount of calmodulin is added.

5. The protein state conversion method of claim 1, wherein calcineurin is used as said serine/threonine protein phosphatase 2B.

6. The protein state conversion method of claim 5, wherein a certain amount of calmodulin is added.

7. A method to convert the state of muscle proteins containing actin, myosin and twitchin, in which the state is converted from a relaxed state, where said actin does not bind to said myosin or the filaments containing myosin, to a catch state, where said actin binds to said myosin or the filaments containing myosin and the binding can sustain a certain tension, by using a serine/threonine protein phosphatase 2B dephosphorylating twitchin, and also the state is inversely converted from said catch state to said relaxed state by using a protein kinase A phosphorylating twitchin, and thus the state can be reversibly converted between the catch and the relaxed states.

8. The protein state conversion method of claim 7, wherein the conversion to said catch state is carried out at approximately 10⁻⁵M free Ca²⁺.

9. The protein state conversion method of claim 8, wherein calcineurin is used as a said serine/threonine protein phosphatase 2B.

10. The protein state conversion method of claim 9, wherein a certain amount of calmodulin is added.

11. The protein state conversion method of claim 7, wherein calcineurin is used as said serine/threonine protein phosphatase 2B.

12. The protein state conversion method of claim 11, wherein a predetermined amount of calmodulin is added.

13. A method of converting a state of actin and myosin and/or filaments containing myosin from a relaxed state where actin does not bind to myosin and/or filaments containing myosin to a catch state comprising;
applying an effective amount of serine/threonine protein phosphatase 2B dephosphorylating twitchin to the actin and myosin and/or filaments containing myosin to provide a catch state.

14. The method of claim 13 further applying an effective amount of protein kinase A phosphorylating twitchin to the actin and myosin and/or filaments containing myosin to convert the catch state to a relax state.
